(19) United States
(12) Patent Application Publication
(10) Pub. No.: US 2021/0315471 A1
(43) Pub. Date: Oct. 14, 2021

(54) METHOD AND DEVICE FOR CORRECTING A BLOOD PRESSURE MEASUREMENT CARRIED OUT AT A MEASUREMENT LOCATION

(71) Applicant: PULSION MEDICAL SYSTEMS SE, Feldkirchen (DE)

(72) Inventors: Thomas THALMEIER, Dorfen (DE); Roman GRULER, Munich (DE)

(21) Appl. No.: 17/272,206

(22) PCT Filed: Aug. 27, 2019
(86) PCT No.: PCT/EP2019/072845

§ 371 Date: Feb. 26, 2021

(30) Foreign Application Priority Data
Aug. 29, 2018 (DE) 10 2018 006 844.8

(51) Int. Cl. A61B 5/022; A61B 5/021; A61B 5/00; A61B 5/0225

(52) U.S. Cl. CPC A61B 5/02241; A61B 5/02141; A61B 5/7405; A61B 5/742; A61B 2560/029; A61B 5/02255; A61B 2560/0257; A61B 2560/0223; A61B 5/7271

(57) ABSTRACT

To calibrate a differential pressure measurement to a geodetic height difference of (approximately) zero, a reference position determination part is led close up to the blood pressure measuring device where a calibration signal is triggered if a minimum distance is not reached between two respective points defined at the reference position determination part and a blood pressure measuring device. A control device integrated in or connected to the blood pressure measuring device performs the calibration measurement in response to the trigger signal. The triggering means are implemented, e.g., as an RFID tag on the reference position determination part-side and an associated near-field communication reader on the blood pressure measuring device-side. If the near-field communication reader detects the correctly encoded RFID tag in the reference position determination part, it triggers the calibration signal.

### TECHNICAL FIELD

The present invention relates to a method for correcting a blood pressure measurement conducted at a measuring position which has a geodetic height difference from a reference position defined by the position of a patient's heart.

### PRIOR ART

The (particularly arterial) blood pressure of a patient is one of the most important measured variables in medical engineering and the known associated measuring equipment, both invasive and non-invasive, is extremely diverse. This applies above all to measuring equipment for continuous monitoring of blood pressure over a longer period of time, for example in intensive care medicine, but also in emergency medicine and during surgical interventions.

For reasons of good accessibility, the blood pressure measuring device is often attached to limbs of the patient, for example an applanation tonometric sensor in the radial artery on the forearm or a photoplethysmographically operated finger sensor based on the so-called “vascular unloading technique” according to Peňáz. Such pressure measuring devices are for example known from U.S. Pat. Nos. 4,406,289, 4,524,777, 4,726,382, WO 2010/050798 A1, WO 2000/059369 A1, WO 2011/045138 A1, WO 2011/051819 A1, WO 2011/051822 A1, WO 2012/032413 A1, and WO 2017/143366 A1.

Pressure sensors connected to a patient's artery via a liquid column using a catheter and hose system for invasive measurements can also be connected to a limb of the patient, for example by means of adhesive tape or suture, or fastened at a slight distance from the patient.

In all of the cases mentioned, the pressure measured locally at the measuring position changes depending on the geodetic height difference between the measuring position and the position of the patient's heart. For example, arterial blood pressure measured at a stationary finger or wrist changes when the patient is moved from a lying down to a half-sitting position or vice versa. Likewise, the arterial blood pressure measured at a finger or wrist changes if the hand is raised or lowered while the patient's position is otherwise unchanged.

In order to correct respective changes in position during continuous blood pressure measurements, such that the blood pressure monitoring or the blood pressure-based patient monitoring remains largely unaffected by changes in the geodetic height difference between the measuring position and the patient's heart, devices are known from the prior art which measure a static differential pressure between the blood pressure measuring device located at the measuring position and a reference position determination part which is attached to a reference position defined by the position of the patient's heart, for example affixed to the chest. For example, EP 0 465 345 A1 discloses a plethysmographic blood pressure sensor with a finger cuff, wherein the hydrostatic pressure difference to the heart is measured by means of a liquid column between a ventilated liquid reservoir of a chest unit attached close to the heart and a pressure sensor in the area of the finger cuff.

U.S. Pat. No. 4,779,626 describes a differential pressure sensor on a finger cuff with a hydraulic connection to a liquid reservoir near the heart, which is designed as a flexible bag with a protective housing.

WO 2006/020917 A1 discloses a system with at least one blood pressure sensor (invasive or non-invasive) and another pressure sensor which is connected to a calibration device, which may be a liquid reservoir.

U.S. Pat. No. 5,957,853 describes an altitude reference system with a measuring pressure sensor for measuring the blood pressure and a reference pressure sensor.

Such devices usually must be calibrated before use. In electronic measuring systems, this usually takes place in that the reference position determination part is brought as closely as possible to the same geodetic height before being attached to the reference position, and the zero point adjustment is then triggered manually by the medical staff, for example using a button or a graphic user interface. To this end, either the respective operating element, such as a touchscreen, must be located directly next to the patient, or two people are required who at the same time bring the reference position determination part into the position suitable for calibration and trigger the zero point adjustment. Furthermore, calibration errors can occur if the reference position determination part is not held with sufficient care in the correct position for calibration.

### SUMMARY OF THE INVENTION

In view of the calibration problems that exist with conventional systems, it is the object of the present invention to simplify the calibration of systems for taking into account a geodetic differential pressure between the measuring position of the blood pressure measurement and a reference position near the heart, and to make it as reliable as possible.

According to one aspect of the present invention, this object is achieved by a method according to claim 1.

According to another aspect, a device according to the claim is provided to achieve the underlying object of the invention.

Preferred embodiments of the invention can be implemented according to one of the dependent claims.

The present invention relates to a method for correcting a blood pressure measurement conducted at a measuring position having a geodetic height difference from a reference position defined by the position of a patient's heart, wherein
a reference position determination part is placed substantially at the geodetic height of a blood pressure measuring device located at the measuring position,
a calibration measurement to establish a zero point for a static pressure difference between the reference position and the measuring position is conducted while the reference position determination part is positioned substantially at the geodetic height of the blood pressure measuring device located at the measuring position,
the reference position determination part is placed at the reference position,
the static pressure difference between the measuring position and the reference position is determined while the reference position determination part is placed at the reference position,
the blood pressure measurement is corrected using the determined static pressure difference.
   The calibration measurement is triggered by a trigger signal exchanged between the reference position determination part and the blood pressure measuring device, the trigger signal being triggered automatically by placing the reference position determination part in the immediate vicinity of the blood pressure measuring device.

Accordingly, the invention also provides a device for correcting a blood pressure measurement conducted at a measuring position having a geodetic height difference from a reference position defined by the position of a patient's heart, the device comprising:
a blood pressure measuring device for placement at the measuring position and for conducting blood pressure measurements,
differential pressure determination means for determining a static pressure difference between a current position of the reference position determination part and a current position of the blood pressure measuring device,
calibration means for conducting a calibration measurement to establish a zero point for the static pressure difference,
correcting means for correcting the blood pressure measurement using the determined static pressure difference, and
triggering means for automatically triggering the calibration measurement when the reference position determination part is placed in the immediate vicinity of the blood pressure measuring device.

Placed substantially at the geodetic height of the blood pressure measuring device located at the measuring position preferably means a geodetic height deviation of 30 millimeters or less, preferably 20 millimeters or less.

Immediate vicinity means a distance between a first defined point on the blood pressure measuring device and a second defined point on the reference position determination part of a maximum of 30, preferably a maximum of 20 millimeters.

According to a particularly advantageous embodiment, the trigger signal is triggered inductively, preferably by means of near-field communication (NFC), and/or magnetically, for example using a reed switch in the blood pressure measuring device, which switch reacts to a magnet arranged in the reference position determination part.

To implement near-field communication, technology known per se can advantageously be used, for example from the field of wireless payment systems or access controls. Thus, according to a preferred embodiment, the reference position determination part can be equipped with a passive HF-RFID tag, which can be read out by a reader integrated in the blood pressure measuring device when the distance falls below a threshold value.

Particularly when using near-field communication, identification means can therefore advantageously be provided for identifying a coding of the reference position determination part. For example, said passive HF-RFID tag can store a code by means of which the associated reader in the blood pressure measuring device identifies the reference position determination part. It can thus be ensured that only reference position determination parts of a suitable specification are used, and the device outputs a warning if an incorrectly specified reference position determination part is connected and/or blocks the function.

For technical reasons, there is often a specific distance between the measuring position and the triggering means on the side of the blood pressure measuring device. If the position of the blood pressure measuring device is changed in such a way that a plane that runs through the measuring position and the triggering means on the side of the blood pressure measuring device is tilted relative to the horizontal, there is also a shift in the geodetic height of the triggering means on the side of the blood pressure measuring device relative to the measuring position.

A change in position and/or the spatial orientation of the blood pressure measuring device relative to the horizontal is therefore preferably detected by means of suitable position change sensor means. If the position change sensor means detect that the geodetic height difference between the position change sensor means and the measuring position exceeds a permissible level (i.e., a threshold value), a warning or a respective correction value can be output. Position change sensor means can be implemented by means of position sensors or acceleration sensors, as is known, for example, from the prior art for determining the spatial orientation of smartphones.

The invention can be used particularly advantageously if the blood pressure measuring device has a finger sensor, since it is precisely here that the geodetic height difference between the heart and the measuring position can be particularly large and can easily be changed.

Particularly, the blood pressure measuring device can advantageously comprise the following:
a radiation source for emitting near-infrared light into a finger through an optical emission surface,
a photodetector for detecting a portion of the near-infrared light captured by an optical collector surface and not absorbed in the finger,
a cuff for receiving the finger, which cuff is assigned to the finger sensor and can be filled with a fluid, and
a pressure regulating system for regulating a fluid pressure in the cuff as a function of the detected non-absorbed portion of the near-infrared light.
   A blood pressure measuring device equipped in this way can determine the arterial blood pressure in the finger according to the so-called “vascular unloading technique.”

The blood pressure measuring device preferably has a base part and a cuff part comprising the cuff, which cuff part can be connected to the base part without tools and can be separated from the base part without tools, and wherein the triggering means are at least partially arranged in the base part. By separating the base and cuff parts, the cuff part, which is mainly in contact with the patient, can be hygienically designed as a disposable item, while the complex measuring and pressure control equipment can mainly be accommodated in the reusable base part. The reference position determination part can also advantageously be designed as a disposable item.

According to a preferred embodiment, the device has notification means for visual and/or acoustic notification of a user that a calibration measurement has taken place. Medical personnel can ensure that the system has been calibrated successfully, for example, by means of a light-emitting diode arranged on the blood pressure measuring device, a tone generator housed in the blood pressure measuring device, or via another output device, for example the display of a patient monitor connected to the blood pressure measuring device. The user initiating the calibration can thus particularly determine that or when the reference position determination part was brought close enough to the blood pressure measuring device to automatically trigger the calibration measurement.

According to a preferred embodiment, the device comprises switching means for activating and deactivating the triggering means. The device can thus to a certain extent be “focused” before use.

In principle, every variant of the invention described or indicated in the context of the present application can be particularly advantageous, depending on the economic, technical, and possibly medical conditions in each individual case. Unless otherwise stated, or as far as technically feasible in principle, individual features of the described embodiments can be exchanged or combined with one another and with features known per se from the prior art.

Particularly, the equipment used to determine the static pressure difference between the measuring position and the reference position can in principle be implemented as known from prior art.

The invention is explained in more detail below by way of example with reference to the accompanying schematic drawings. The drawings are not to scale; particularly, for reasons of clarity, the relationships between the individual dimensions do not necessarily correspond to the dimensional relationships in actual technical implementations. Corresponding elements are identified by the same reference numerals in the individual figures.

### BRIEF DESCRIPTION OF THE FIGURES

FIG. 1 schematically shows the overall arrangement of a device according to the invention, wherein the position of the reference position determination part is shown once in the measurement configuration (solid line) and once in the calibration configuration (dashed line).

FIG. 2 schematically shows another device according to the invention, similar to the one shown in FIG. 1, in a calibration configuration.

FIG. 3a shows the pressure measuring device of the device of FIG. 2 on a horizontal support.

FIG. 3b shows the pressure measuring device of the device of FIG. 2 in a tilted state.

FIG. 4a shows a side view of the pressure measuring device shown in FIG. 3a.

FIG. 4b shows a front view of the pressure measuring device of FIG. 4a (from the left in FIG. 4a).

FIG. 5 shows an enlarged view of FIG. 4b with schematically sketched photoplethysmographic components.

### DETAILED DESCRIPTION OF ADVANTAGEOUS EXEMPLARY EMBODIMENTS

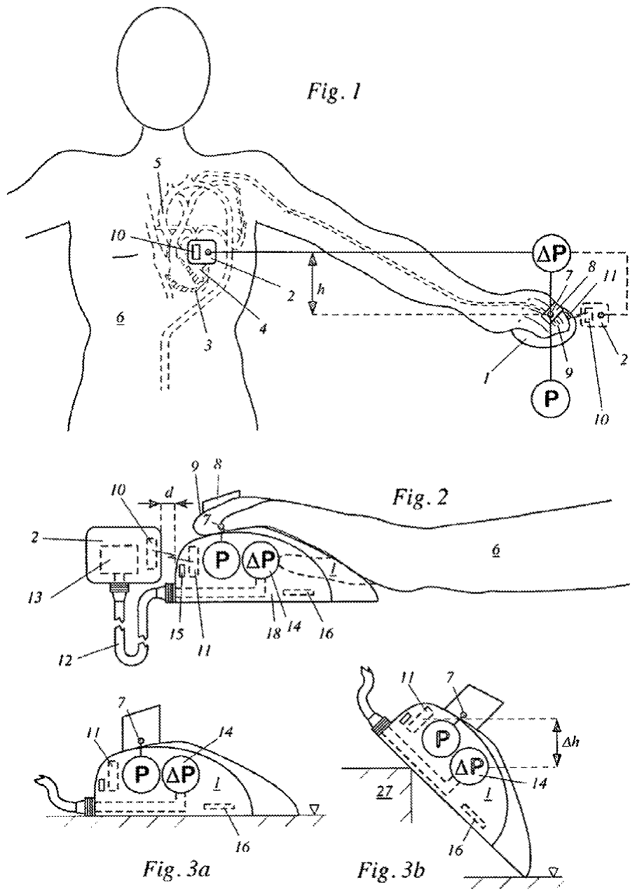

The device shown in FIG. 1 comprises a blood pressure measuring device 1 and a reference position determination part 2 which in the measurement configuration is attached near the heart 3, as close as possible to the left ventricle 4 or aorta 5 of the patient 6, which is to be regarded as the reference position, for example affixed to the skin of the patient 6. As is basically known from prior art, the device measures a pressure difference ΔP resulting from the geodetic height difference h between the reference position determination part 2 and the blood pressure measuring device 1 located near the measuring position, for example by means of a liquid column between the blood pressure measuring device 1 and the reference position determination part 2 using a pressure sensor arranged on the side of the blood pressure measuring device or pressure sensors arranged on both ends of the liquid column, in order to correct the blood pressure P measured by the blood pressure measuring device 1.

In the example shown, the measuring position 7 is located on a finger 9 of the patient 6 that is surrounded by a cuff part 8.

To calibrate the differential pressure measurement to a geodetic height difference of (approximately) zero, the reference position determination part 2 is brought so close to the blood pressure measuring device 1 that a calibration signal is triggered if a minimum distance between two respective points defined on the reference position determination part 2 and the blood pressure measuring device 1 is not reached. The points can advantageously be marked on the respective housings of the reference position determination part 2 and the blood pressure measuring device 1, such that the operating personnel can simply be instructed to bring the marked points closer together for calibration, e.g. to a distance of at most 3 cm, at most 2 cm, or at most 1 cm as the threshold value from which the triggering means respond with the output of a triggering signal.

A control device (not shown) integrated in the blood pressure measuring device 1 or connected thereto, which preferably has a microprocessor or microcontroller, conducts the calibration measurement in response to the trigger signal.

As illustrated in FIG. 2, the triggering means in the example described are implemented as an RFID tag 10 on the side of the reference position determination part and an associated near-field communication reader 11 on the side of the blood pressure measuring device. If the near-field communication reader 11 in the blood pressure measuring device 1 detects the correctly encoded RFID tag in the reference position determination part 2, it triggers the calibration signal. Successful triggering of the calibration signal and/or completion of the calibration measurement is indicated to the user by the light-emitting diode unit (LED unit) 15.

The reference position determination part (2) to be attached to the patient is preferably designed as a disposable article, meeting the associated increased sterility requirements. It is connected to the blood pressure measuring device via the hose connection 12 (shown by a dashed line in FIG. 2), such that there is a coherent liquid column between a liquid reservoir 13 in the reference position determination part 2 and a correction pressure sensor 14 in the blood pressure measuring device 1. The correction pressure sensor can be a commercially available sensor, for example a piezoelectric, piezoresistive, inductive, or capacitive pressure sensor.

As illustrated in FIGS. 3a and 3b, deviations can arise when the blood pressure measuring device 1 is tilted, for example by partially resting on a raised object 27. In the example shown, tilting in conjunction with the distance between the near-field communication reader 11 and the correction pressure sensor 14 results in a geodetic height difference Δh that falsifies the measurement correction if calibration is carried out in the tilted position. Depending on the installation conditions, tilting during blood pressure measurement can also result in a changed geodetic height difference between the measuring position 7 of the blood pressure measurement and the correction pressure sensor 14. In order to correct such falsifications arithmetically, or to output a warning signal when a threshold tilt angle relative to the horizontal is exceeded, one or more position or position change sensors 16 can be provided in the blood pressure measuring device 1.

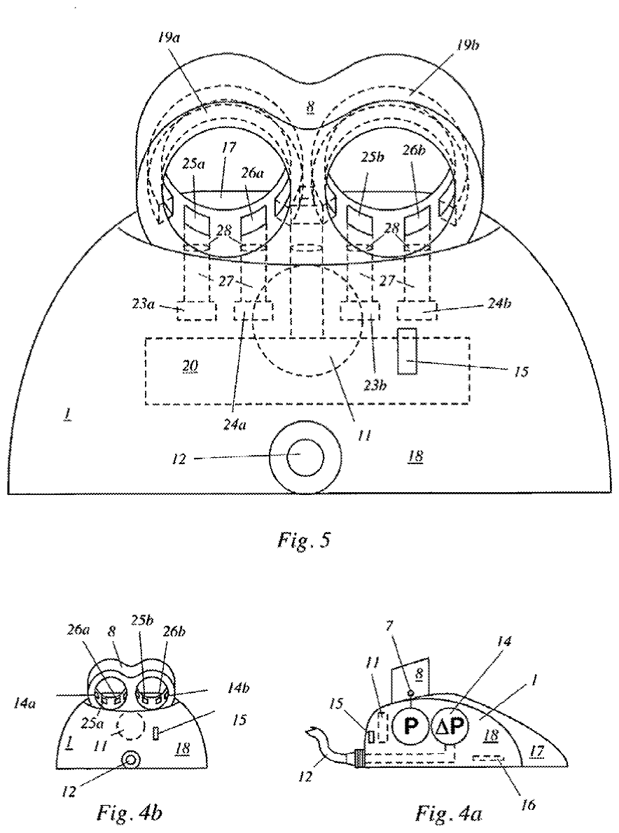

In the example shown, the blood pressure measuring device 1 itself is designed as a photoplethysmographic measuring system which functions in accordance with the so-called “vascular unloading technique.” Measurement components can basically be implemented similar to the prior art mentioned at the outset. Essential components of the exemplary embodiment described are sketched in FIG. 4b and particularly FIG. 5, which show a front view of the pressure measuring device 1 shown in a side view in FIGS. 3a and 4a (from the left in FIGS. 3a and 4a). Elements arranged within the housing are indicated by dashed lines.

The cuff part 8 is designed to accommodate two fingers, which makes it possible to measure alternately on both fingers. For hygienic reasons, the cuff part 8, together with the palm rest 17, is designed as a disposable item, which is attached to the reusable base part 18 in a detachable manner by means of a plug-in connection.

The two inflatable finger cuffs 19a, 19b are connected to the pressure generation and pressure control system 20 via a distributor 21 and a connection 22 at the interface between the cuff part 8 and the base part 18. In alternative embodiments, the finger cuffs 19a, 19b can also be connected separately to a (optionally, a respective) pressure generation and pressure control system 20 and can thus be controlled separately.

A light source 23a, 23b for near-infrared light and a photodetector 24a, 24b are provided for each of the two fingers, for example as a light-emitting diode, which is connected via a respective so-called light pipe 27, i.e., a light guide not designed as a fiber bundle, to an associated optical emission surface 25a, 25b or optical collector surface 26a, 26b for coupling emitted light into the finger tissue or coupling non-absorbed light out from the finger tissue. At the interface between the cuff part 8 and the base part 18, the cuff-part-side and base-part-side sections of the light pipes 27 are connected to one another via separable optical contact points 28.

The pressure generation and pressure control system 20 regulates the cuff pressure in accordance with the signal received by one of the photodetectors 24a, 24b, such that the portion of the near-infrared light emitted by the associated light source 23a, 23b that is not absorbed in the corresponding finger remains as constant as possible, that is, a respective pulsatile portion of the arterial blood pressure is generated, such that the blood volume present in the respective finger area (and plethysmographically detected by the respective light source/detector pair 23a, 24a or 23b, 24b) remains approximately constant. The counterpressure in the cuffs 19a, 19b, regulated accordingly by the pressure generation and pressure control system 20, is detected as a blood pressure measurement signal by a sensor in the pressure generation and pressure control system 20 and corrected using the geodetically determined pressure difference to the reference position measured by the correction pressure sensor 14. The corrected value can be output to a patient monitor via a suitable electronic interface.

1. A method of correcting a blood pressure measurement conducted at a measuring position having a geodetic height difference from a reference position defined by the position of a patient's heart, the method comprising:
placing a reference position determination part substantially at the geodetic height of a blood pressure measuring device located at the measuring position;
conducting a calibration measurement to establish a zero point for a static pressure difference between the reference position and the measuring position while the reference position determination part is positioned substantially at the geodetic height of the blood pressure measuring device located at the measuring position;
placing the reference position determination part at the reference position;
determining the static pressure difference between the measuring position and the reference position while the reference position determination part is placed at the reference position; and
correcting the blood pressure measurement using the determined static pressure difference,
wherein the calibration measurement is triggered by a trigger signal exchanged between the reference position determination part and the blood pressure measuring device,
wherein the trigger signal is automatically triggered by placing the reference position determination part in an immediate vicinity of the blood pressure measuring device.

2. The method according to claim 1, wherein immediate vicinity means a distance between a first defined point on the blood pressure measuring device and a second defined point on the reference position determination part of a maximum of 30 millimeters.

3. The method according to claim 1, wherein the trigger signal is at least one of inductive by means of near-field communication, is triggered magnetically.

4. The method according to claim 1, wherein a change in at least one of the position or the spatial orientation of the blood pressure measuring device is detected relative to the horizontal.

5. A device for correcting a blood pressure measurement conducted at a measuring position having a geodetic height difference from a reference position defined by the position of a patient's heart, the device comprising:
a reference position determination part configured for attachment to the reference position;
a blood pressure measuring device configured for placement at the measuring position and for conducting blood pressure measurements;
differential pressure determination means for determining a static pressure difference between a current position of the reference position determination part and a current position of the blood pressure measuring device;
calibration means for conducting a calibration measurement to establish a zero point for the static pressure difference;
correcting means for correcting the blood pressure measurement using the determined static pressure difference; and
triggering means for automatically triggering the calibration measurement when the reference position determination part is placed in the immediate vicinity of the blood pressure measuring device.

6. The device according to claim 5, wherein immediate vicinity means a distance between a first defined point on the blood pressure measuring device and a second defined point on the reference position determination part of a maximum of 30 millimeters.

7. The device according to claim 5, wherein the triggering means comprise at least one of (i) means for detecting electromagnetic induction between first induction means arranged in the reference position determination part and second induction means arranged in the blood pressure measuring device, or (ii) means for detecting magnetic forces between first magnetic means in the blood pressure measuring device and second magnetic means in the reference position determination part.

8. The device according to claim 7, wherein the triggering means comprise near-field communication means for performing near-field communication between the reference position determination part and the blood pressure measuring device.

9. The device according to claim 8, wherein the near-field communication means comprises identification means for identifying a code of the reference position determination part.

10. The device according to claim 5, wherein the blood pressure measuring device comprises position change sensor means for detecting a change at least one of in position or spatial orientation of the blood pressure measuring device relative to the horizontal.

11. The device according to claim 5, wherein the blood pressure measuring device has a finger sensor.

12. The device according to claim 11, wherein the blood pressure measuring device comprises:
a radiation source for emitting near-infrared light into a finger through an optical emission surface;
a photodetector for detecting a portion of the near-infrared light captured by an optical collector surface and not absorbed in the finger;
a cuff for receiving the finger, which cuff is assigned to the finger sensor and can be filled with a fluid; and
a pressure regulating system for regulating a fluid pressure in the cuff as a function of the detected non-absorbed portion of the near-infrared light.

13. The device according to claim 12, wherein the blood pressure measuring device has a base part and a cuff part comprising the cuff, which cuff part can be connected to the base part without tools and can be separated from the base part without tools, and wherein the triggering means are at least partially arranged in the base part.

14. The device according to claim 5, further comprising notification means for visual and/or acoustic notification of a user that a calibration measurement has taken place.

15. The device according to claim 5, further comprising switching means for activating and deactivating the triggering means.
